SEQUENCE LISTING (4 sequences)

SEQ ID NO: 1 - DNA, 831 bp, Homo sapiens
atgagaccttggaagagcagcaccatcattatgctgaacctggcctgcacagatctgctgtatctgaccagcctccccttcctgattcactactatgccagtggcgaaaactggatctttggagatttcatgtgtaagtttatccgcttcagcttccatttcaacctgtatagcagcatcctcttcctcacctgtttcagcatcttccgctactgtgtgatcattcacccaatgagctgcttttccattcacaaaactcgatgtgcagttgtagcctgtgctgtggtgtggatcatttcactggtagctgtcattccgatgaccttcttgatcacatcaaccaacaggaccaacagatcagcctgtctcgacctcaccagttcggatgaactcaatactattaagtggtacaacctgattttgactgcaactactttctgcctccccttggtgatagtgacactttgctataccacgattatccacactctgacccatggactgcaaactgacagctgccttaagcagaaagcacgaaggctaaccattctgctactccttgcattttacgtatgttttttacccttccatatcttgagggtcattcggatcgaatctcgcctgctttcaatcagttgttccattgagaatcagatccatgaagcttacatcgtttctagaccattagctgctctgaacacctttggtaacctgttactatatgtggtggtcagcgacaactttcagcaggctgtctgctcaacagtgagatgcaaagtaagcgggaaccttgagcaagcaaagaaaattagttactcaaacaacccttga

SEQ ID NO: 2 - protein, 276 aa, Homo sapiens [sequence omitted]

SEQ ID NO: 3 - DNA, 1014 bp, Homo sapiens
atgaatgagccactagactatttagcaaatgcttctgatttccccgattatgcagctgcttttggaaattgcactgatgaaaacatcccactcaagatgcactacctccctgttatttatggcattatcttcctcgtgggatttccaggcaatgcagtagtgatatccacttacattttcaaaatgagaccttggaagagcagcaccatcattatgctgaacctggcctgcacagatctgctgtatctgaccagcctccccttcctgattcactactatgccagtggcgaaaactggatctttggagatttcatgtgtaagtttatccgcttcagcttccatttcaacctgtatagcagcatcctcttcctcacctgtttcagcatcttccgctactgtgtgatcattcacccaatgagctgcttttccattcacaaaactcgatgtgcagttgtagcctgtgctgtggtgtggatcatttcactggtagctgtcattccgatgaccttcttgatcacatcaaccaacaggaccaacagatcagcctgtctcgacctcaccagttcggatgaactcaatactattaagtggtacaacctgattttgactgcaactactttctgcctccccttggtgatagtgacactttgctataccacgattatccacactctgacccatggactgcaaactgacagctgccttaagcagaaagcacgaaggctaaccattctgctactccttgcattttacgtatgttttttacccttccatatcttgagggtcattcggatcgaatctcgcctgctttcaatcagttgttccattgagaatcagatccatgaagcttacatcgtttctagaccattagctgctctgaacacctttggtaacctgttactatatgtggtggtcagcgacaactttcagcaggctgtctgctcaacagtgagatgcaaagtaagcgggaaccttgagcaagcaaagaaaattagttactcaaacaacccttga

SEQ ID NO: 4 - protein, 337 aa, Homo sapiens
MNEPLDYLANASDFPDYAAAFGNCTDENIPLKMHYLPVIYGIIFLVGFPGNAVVISTYIFKMRPWKSSTIIMLNLACTDLLYLTSLPFLIHYYASGENWIFGDFMCKFIRFSFHFNLYSSILFLTCFSIFRYCVIIHPMSCFSIHKTRCAVVACAVVWIISLVAVIPMTFLITSTNRTNRSACLDLTSSDELNTIKWYNLILTATTFCLPLVIVTLCYTTIIHTLTHGLQTDSCLKQKARRLTILLLLAFYVCFLPFHILRVIRIESRLLSISCSIENQIHEAYIVSRPLAALNTFGNLLLYVVVSDNFQQAVCSTVRCKVSGNLEQAKKISYSNNP

(19) United States
(12) Patent Application Publication
(10) Pub. No.: US 2002/0065215 A1
(43) Pub. Date: May 30, 2002

(54) Polypeptide

(76) Inventors: Steven M. Foord, Stevenage (GB); Diane Michele Ignar, Durham, NC (US)

Correspondence Address: DAVID J LEVY, CORPORATE INTELLECTUAL PROPERTY, GLAXOSMITHKLINE, FIVE MOORE DR., PO BOX 13398, DURHAM, NC, 27709-3398, US

(21) Appl. No.: 09/943,798

(22) Filed: Aug. 31, 2001

(30) Foreign Application Priority Data
Sep. 1, 2000 (GB) 0021524.4
Sep. 6, 2000 (GB) 0021894.1
Sep. 25, 2000 (GB) 0023444.3

(51) Int. Cl. A61K 38/00; C12Q 1/00; A61K 31/70; A61K 39/395

(52) U.S. Cl. 514/2; 514/44; 435/4; 424/130.1

(57) ABSTRACT

The present invention relates to an isolated P2Y-like receptor polypeptide, polynucleotides encoding the same and methods related thereto.

### RELATED APPLICATIONS

The present invention claims priority from Great Britain application number GB0021524.4 filed Sep. 1, 2000, GB0021894.1 filed Sep. 6, 2000 and GB0023444.3 filed Sep. 25, 2000

### FIELD OF THE INVENTION

The present invention relates to P2Y receptor-like polypeptides.

### BACKGROUND OF THE INVENTION

G-protein coupled receptors (GPCRs) are a super-family of membrane receptors that mediate a wide variety of biological functions. Upon binding of extracellular ligands, GPCRs interact with a specific subset of heterotrimeric G proteins that can, in their activated forms, inhibit or activate various effector enzymes and/or ion channels. All GPCRs are predicted to share a common molecular architecture consisting of seven transmembrane helices linked by alternating intracellular and extracellular loops. The extracellular receptor surface has been shown to be involved in ligand binding whereas the intracellular portions are involved in G protein recognition and activation.

The P2Y family of purinergic receptors comprise 5 known human receptors and a large number of related orphan receptors. Tissue distribution analysis for many of these receptors indicates a role in respiratory and immune diseases. For example, P2Y2 controls nucleotide-regulated mucus secretion from goblet and sub-mucosal glands in the lung and is thought to be a therapeutic target for bronchitis. The P2Y1 receptor has potential roles in the brain, lung and immune system based on immunohistochemistry data. Many of these receptors are expressed in human immune cells. For example, P2YGW2 and P2Y10 are highly expressed in dendritic cells, P2Y6 in T and B cells, and P2YGW1 (another GW proprietary sequence) in monocytes.

### SUMMARY OF THE INVENTION

A novel P2Y receptor-like polypeptide, referred to herein as HIPHUM 0000037, is now provided. The expression of HIPHUM 0000037 is shown to be ubiquitous (FIG. 1). Expression is seen in some immune cells (monocytes and dendritic cells), adipose tissue and in endothelial cells (FIG. 3). Growth factor stimulation of bone marrow cells upregulated the expression of this receptor and expression was 15 fold higher in osteoarthritic than normal cartilage and was down-regulated in differentiated osteoblasts (FIG. 3). Receptor expression was up-regulated in 3 colon tumors vs. paired normal tissues (FIG. 2). Thyroid and urinary bladder expression was also higher than other tissues. The novel P2Y receptor-like polypeptide is a screening target for the identification and development of novel pharmaceutical agents, including modulators of P2Y receptor activity. These agents may be used in the treatment and/or prophylaxis of disorders such as colon cancer or diseases of immunity or inflammation such as asthma, COPD, Crohn's disease, irritable bowel syndrome, gastroenteritis and colitis, inflammatory bowel syndrome, ulcerative colitis, rheumatoid arthritis, viral diseases, bacterial infections, autoimmune diseases, dermatitis, glomerulonephritis allergies, allergic rhinitis, inflammatory pain and general inflammation such as tendonitis, polymyositis, or prostatitis.

Accordingly, the present invention provides an isolated P2Y receptor-like polypeptide comprising

(i) the amino acid sequence of SEQ ID NO: 2 or SEQ ID NO: 4;

(ii) a variant thereof which shows immunomodulatory activity; or

(iii) a fragment of (i) or (ii) which shows immunomodulatory activity.

According to another aspect of the invention there is provided a polynucleotide encoding a polypeptide of the invention which polynucleotide includes a sequence comprising:

(a) the nucleic acid sequence of SEQ ID NO: 1 or SEQ ID NO: 3 and/or a sequence complementary thereto;

(b) a sequence which hybridises under stringent conditions to a sequence as defined in (a);

(c) a sequence that is degenerate as a result of the genetic code to a sequence as defined in (a) or (b); or

(d) a sequence having at least 60% identity to a sequence as defined in (a), (b) or (c).

The invention also provides:

an expression vector which comprises a polynucleotide of the invention and which is capable of expressing a polypeptide of the invention;

a host cell comprising an expression vector of the invention;

a method of producing a polypeptide of the invention which method comprises maintaining a host cell of the invention under conditions suitable for obtaining expression of the polypeptide and isolating the said polypeptide;

an antibody specific for a polypeptide of the invention;

a method for identification of a substance that modulates P2Y receptor-like activity and/or expression, which method comprises contacting a polypeptide, polynucleotide, expression vector or host cell of the invention with a test substance and determining the effect of the test substance on the activity and/or expression of the said polypeptide or the polypeptide encoded by the said polynucleotide, thereby to determine whether the test substance modulates P2Y receptor-like activity and/or expression;

a compound which or modulates P2Y receptor activity and which is identifiable by the method referred to above;

a method of treating a subject having a disorder that is responsive to P2Y receptor-like stimulation or modulation, which method comprises administering to said subject an effective amount of substance of the invention; and

use of a substance that stimulates or modulates P2Y receptor activity in the manufacture of a medicament for the treatment or prophylaxis of a disorder that is responsive to stimulation or modulation of P2Y receptor-like activity.

Preferably the disorder is selected from colon cancer or diseases of immunity or inflammation such as asthma, COPD, Crohn's disease, irritable bowel syndrome, gastroenteritis and colitis, inflammatory bowel syndrome, ulcerative colitis, rheumatoid arthritis, viral diseases, bacterial infections, autoimmune diseases, dermatitis, glomerulonephritis allergies, allergic rhinitis, inflammatory pain and general inflammation such as tendonitis, polymyositis, or prostatitis.

### BRIEF DESCRIPTION OF THE SEQUENCES

SEQ ID NO: 1 shows the nucleotide and amino acid sequences of human protein HIPHUM 0000037.

SEQ ID NO: 2 is the amino acid sequence alone of HIPHUM 0000037.

SEQ ID NO: 3 shows the nucleotide and amino acid sequences of a variant of the human protein HIPHUM 0000037 sequence of SEQ ID NO: 1, comprising an extended 5′ region.

SEQ ID NO: 4 is the amino acid sequence alone encoded by the nucleotide sequence of SEQ ID NO: 3.

### BRIEF DESCRIPTION OF THE FIGURES

FIG. 1 shows the relative abundance of HIPHUM 0000037 mRNA in a number of human tissues.

FIG. 2 shows the relative abundance of HIPHUM 0000037 mRNA in normal and tumor pairs from the colon, lung and breast.

FIG. 3 shows the relative abundance of HIPHUM 0000037 in a variety of immune cells, in lung tissue from normal, asthmatic or chronic obstructive pulmonary disease (COPD) subjects, in normal or growth factor stimulated endothelial cells and bone marrow, in normal or osteoarthritic (OA) cartilage, in normal or rheumatoid arthritic (RA) synovium and in differentiated or undifferentiated osteoblasts.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

The present invention relates to a human P2Y-like receptor, referred to herein as HIPHUM 0000037, and variants thereof Sequence information for HIPHUM 0000037 is provided in SEQ ID NOs: 1 and 3 (nucleotide and amino acid) and in SEQ ID NOs: 2 and 4. A polypeptide of the invention thus consists essentially of the amino acid sequence of SEQ ID NO: 2 or SEQ ID NO: 4 or of a variant of that sequence, or of a fragment of either thereof.

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 50%, e.g. more than 80%, 90%, 95% or 99%, by weight of the polypeptide in the preparation is a polypeptide of the invention. Routine methods, can be employed to purify and/or synthesise the proteins according to the invention. Such methods are well understood by persons skilled in the art, and include techniques such as those disclosed in Sambrook et al, Molecular Cloning: a Laboratory Manual, 2nd Edition, CSH Laboratory Press, 1989, the disclosure of which is included herein in its entirety by way of reference.

The term “variant” refers to a polypeptide which has a same essential character or basic biological functionality as HIPHUM 0000037. The essential character of HIPHUM 0000037 can be defined as follows: HIPHUM 0000037 is a P2Y-like receptor. Preferably a variant polypeptide is one which binds to the same ligand as HIPHUM 0000037. Preferably the polypeptide shows immunomodulatory activity. A polypeptide having a same essential character as HIPHUM 0000037 may be identified by monitoring for a function of the receptor selected from P2Y receptor-like activity or effects on the immune system or immune or inflammatory processes. A variant receptor may be identified by looking for ligand binding. Possible ligands include ATP, ADP, and AMP and derivatives thereof such as 2 methylthio-ATP, 2 methylthio-ADP, homo-ATP, ATPγS, ATPαS, ATPβF, UTP, UDP, αβ, methyl ATP, Ap2A, Ap3A, Ap4A, Ap6A, CTP, CDP, ITP, IDP, GTP, GDP, TTP, TDP, ADP-glucose, CDP-glucose, TDP-glucose, GDP-glucose, UDP-glucose, UDP-galacturonic acid, UDP-glucuronic acid, UDP-N acetylglucosamine, UDP-mannose, UDP-oxylose, ADP-ribose, and/or ATP-ribose.

A full length variant polypeptide is preferably one which includes a seven transmembrane region. Preferably, a full length variant polypeptide may couple to G-protein to mediate intracellular responses.

In another aspect of the invention, a variant is one which does not show the same activity as HIPHUM 0000037 but is one which inhibits a basic function of HIPHUM 0000037. For example, a variant polypeptide is one which inhibits the immunomodulatory activity of HIPHUM 0000037, for example by binding to HIPHUM 0000037 ligand to prevent activity mediated by ligand binding to HIPHUM 0000037.

Typically, polypeptides with more than about 65% identity preferably at least 80% or at least 90% and particularly preferably at least 95% at least 97% or at least 99% identity, with the amino acid sequences of SEQ ID NO: 2 or SEQ ID NO: 4, are considered as variants of the proteins. Such variants may include allelic variants and the deletion, modification or addition of single amino acids or groups of amino acids within the protein sequence, as long as the peptide maintains a basic biological functionality of the HIPHUM 0000037 receptor.

Amino acid substitutions maybe made, for example from 1, 2 or 3 to 10, 20 or 30 substitutions. The modified polypeptide generally retains activity as a P2Y-like or immunomodulatory receptor. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Shorter polypeptide sequences are within the scope of the invention. For example, a peptide of at least 20 amino acids or up to 50, 60, 70, 80, 100, 150 or 200 amino acids in length is considered to fall within the scope of the invention as long as it demonstrates a basic biological functionality of HIPHUM 0000037. In particular, but not exclusively, this aspect of the invention encompasses the situation when the protein is a fragment of the complete protein sequence and may represent a ligand-binding region (N-terminal extracellular domain) or an effector binding region (C-terminal intracellular domain). Such fragments can be used to construct chimeric receptors preferably with another 7-transmembrane receptor, more preferably with another member of the family of P2Y receptors. Such fragments of HIPHUM 0000037 or a variant thereof can also be used to raise anti-HIPHUM 0000037 antibodies. In this embodiment the fragment may comprise an epitope of the HIPHUM 0000037 polypeptide and may otherwise not demonstrate the ligand binding or other properties of HIPHUM 0000037.

Polypeptides of the invention may be chemically modified, e.g. post-translationally modified. For example, they may be glycosylated or comprise modified amino acid residues. They may also be modified by the addition of histidine residues to assist their purification or by the addition of a signal sequence to promote insertion into the cell membrane. Such modified polypeptides fall within the scope of the term “polypeptide” of the invention.

The invention also includes nucleotide sequences that encode for HIPHUM 0000037 or variant thereof as well as nucleotide sequences which are complementary thereto. The nucleotide sequence may be RNA or DNA including genomic DNA, synthetic DNA or cDNA. Preferably the nucleotide sequence is a DNA sequence and most preferably, a cDNA sequence. Nucleotide sequence information is provided in SEQ ID NOs: 1 and 3. Such nucleotides can be isolated from human cells or synthesised according to methods well known in the art, as described by way of example in Sambrook et al, 1989.

Typically a polynucleotide of the invention comprises a contiguous sequence of nucleotides which is capable of hybridizing under selective conditions to the coding sequence or the complement of the coding sequence of SEQ ID NO: 1 or SEQ ID NO: 3.

A polynucleotide of the invention can hybridize to the coding sequence or the complement of the coding sequence of SEQ ID NO: 1 or SEQ ID NO: 3 at a level significantly above background. Background hybridization may occur, for example, because of other cDNAs present in a cDNA library. The signal level generated by the interaction between a polynucleotide of the invention and the coding sequence or complement of the coding sequence of SEQ ID NO: 1 or SEQ ID NO: 3 is typically at least 10 fold, preferably at least 100 fold, as intense as interactions between other polynucleotides and the coding sequence of SEQ ID NO: 1 or SEQ ID NO: 3. The intensity of interaction may be measured, for example, by radiolabelling the probe, e.g. with ³²P. Selective hybridisation may typically be achieved using conditions of medium to high stringency. However, such hybridisation may be carried out under any suitable conditions known in the art (see Sambrook et al, 1989. For example, if high stringency is required suitable conditions include from 0.1 to 0.2× SSC at 60 ° C. up to 65° C. If lower stringency is required suitable conditions include 2× SSC at 60° C.

The coding sequence of SEQ ID NO: 1 or SEQ ID NO: 3 may be modified by nucleotide substitutions, for example from 1, 2 or 3 to 10, 25, 50 or 100 substitutions. The polynucleotide of SEQ ID NO: 1 or SEQ ID NO: 3 may alternatively or additionally be modified by one or more insertions and/or deletions and/or by an extension at either or both ends. A polynucleotide may include one or more introns, for example may comprise genomic DNA. Additional sequences such as signal sequences which may assist in insertion of the polypeptide in a cell membrane may also be included. The modified polynucleotide generally encodes a polypeptide which has a HIPHUM 0000037 receptor activity. Alternatively, a polynucleotide encodes a ligand-binding portion of a polypeptide or a polypeptide which inhibits HIPHUM 0000037 activity. Degenerate substitutions may be made and/or substitutions may be made which would result in a conservative amino acid substitution when the modified sequence is translated, for example as shown in the Table above.

A nucleotide sequence which is capable of selectively hybridizing to the complement of the DNA coding sequence of SEQ ID NO: 1 or SEQ ID NO: 3 will generally have at least 60%, at least 70%, at least 80%, at least 90%, at least 95%, at least 98% or at least 99% sequence identity to the coding sequence of SEQ ID NO: 1 or SEQ ID NO: 3 over a region of at least 20, preferably at least 30, for instance at least 40, at least 60, more preferably at least 100 contiguous nucleotides or most preferably over the full length of SEQ ID NO: 1 or SEQ ID NO: 3.

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul (1993) J. Mol. Evol. 36:290-300; Altschul et al (1990) J. Mol. Biol. 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, 1990). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Any combination of the above mentioned degrees of sequence identity and minimum sizes may be used to define polynucleotides of the invention, with the more stringent combinations (i.e. higher sequence identity over longer lengths) being preferred. Thus, for example a polynucleotide which has at least 90% sequence identity over 25, preferably over 30 nucleotides forms one aspect of the invention, as does a polynucleotide which has at least 95% sequence identity over 40 nucleotides.

The nucleotides according to the invention have utility in production of the proteins according to the invention, which may take place in vitro, in vivo or ex vivo. The nucleotides may be involved in recombinant protein synthesis or indeed as therapeutic agents in their own right, utilised in gene therapy techniques. Nucleotides complementary to those encoding HIPHUM 0000037, or antisense sequences, may also be used in gene therapy.

Polynucleotides of the invention may be used as a primer, e.g. a PCR primer, a primer for an alternative amplification reaction, a probe e.g. labelled with a revealing label by conventional means using radioactive or non-radioactive labels, or the polynucleotides may be cloned into vectors.

Such primers, probes and other fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of the coding sequence of SEQ ID NO: 1 or SEQ ID NO:

The present invention also includes expression vectors that comprise nucleotide sequences encoding the proteins or variants thereof of the invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. 1989.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used as test compounds in the assays of the invention or may be useful in a method of treatment of the human or animal body by therapy.

Preferably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with a origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example in a method of gene therapy.

Promoters and other expression regulation signals may be selected to be compatible with the host cell for which expression is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

Mammalian promoters, such as β-actin promoters, may be used. Tissue-specific promoters are especially preferred. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, adenovirus, HSV promoters (such as the HSV IE promoters), or HPV promoters, particularly the HPV upstream regulatory region (URR). Viral promoters are readily available in the art.

The vector may further include sequences flanking the polynucleotide giving rise to polynucleotides which comprise sequences homologous to eukaryotic genomic sequences, preferably mammalian genomic sequences, or viral genomic sequences. This will allow the introduction of the polynucleotides of the invention into the genome of eukaryotic cells or viruses by homologous recombination. In particular, a plasmid vector comprising the expression cassette flanked by viral sequences can be used to prepare a viral vector suitable for delivering the polynucleotides of the invention to a mammalian cell. Other examples of suitable viral vectors include herpes simplex viral vectors and retroviruses, including lentiviruses, adenoviruses, adeno-associated viruses and HPV viruses. Gene transfer techniques using these viruses are known to those skilled in the art. Retrovirus vectors for example may be used to stably integrate the polynucleotide giving rise to the polynucleotide into the host genome. Replication-defective adenovirus vectors by contrast remain episomal and therefore allow transient expression.

The invention also includes cells that have been modified to express the HIPHUM 0000037 polypeptide or a variant thereof. Such cells include transient, or preferably stable higher eukaryotic cell lines, such as mammalian cells or insect cells, using for example a baculovirus expression system, lower eukaryotic cells, such as yeast or prokaryotic cells such as bacterial cells. Particular examples of cells which may be modified by insertion of vectors encoding for a polypeptide according to the invention is include mammalian HEK293T, CHO, HeLa and COS cells. Preferably the cell line selected will be one which is not only stable, but also allows for mature glycosylation and cell surface expression of a polypeptide. Expression may be achieved in transformed oocytes. A polypeptide of the invention may be expressed in cells of a transgenic non-human animal, preferably a mouse. A transgenic non-human animal expressing a polypeptide of the invention is included within the scope of the invention. A polypeptide of the invention may also be expressed in Xenopus laevis oocytes or melanophores, in particular for use in an assay of the invention.

According to another aspect, the present invention also relates to antibodies, specific for a polypeptide of the invention. Such antibodies are for example useful in purification, isolation or screening methods involving immunoprecipitation techniques or, indeed, as therapeutic agents in their own right.

Antibodies may be raised against specific epitopes of the polypeptides according to the invention. Such antibodies may be used to block ligand binding to the receptor. An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

Antibodies of the invention may be antibodies to human polypeptides or fragments thereof. For the purposes of this invention, the term “antibody”, unless specified to the contrary, includes fragments which bind a polypeptide of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies may be used in a method for detecting polypeptides of the invention in a biological sample, which method comprises:

I providing an antibody of the invention;

II incubating a biological sample with said antibody under conditions which allow for the formation of an antibody-antigen complex; and

III determining whether antibody-antigen complex comprising said antibody is formed.

A sample may be for example a tissue extract, blood, serum and saliva. Antibodies of the invention may be bound to a solid support and/or packaged into kits in a suitable container along with suitable reagents, controls, instructions, etc. Antibodies may be linked to a revealing label and thus may be suitable for use in methods of in vivo HIPHUM 0000037 imaging.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An important aspect of the present invention is the use of polypeptides according to the invention in screening methods. The screening methods may be used to identify substances that bind to P2Y receptors and in particular which bind to HIPHUM 0000037 such as a ligand for the receptor. Screening methods may also be used to identify agonists or antagonists which may modulate P2Y receptor or immunomodulatory activity, inhibitors or activators of HIPHUM 0000037 activity, and/or agents which up-regulate or down-regulate HIPHUM 0000037 expression.

Any suitable format may be used for the assay. In general terms such screening methods may involve contacting a polypeptide of the invention with a test substance and monitoring for binding of the test substance to the polypeptide or measuring receptor activity. A polypeptide of the invention may be incubated with a test substance. Modulation of P2Y receptor-like activity may be determined. In a preferred aspect, the assay is a cell-based assay. Preferably the assay may be carried out in a single well of a microtitre plate. Assay formats which allow high throughput screening are preferred.

Modulator activity can be determined by contacting cells expressing a polypeptide of the invention with a substance under investigation and by monitoring an effect mediated by the polypeptide. The cells expressing the polypeptide may be in vitro or in vivo. The polypeptide of the invention may be naturally or recombinantly expressed. Preferably, the assay is carried out in vitro using cells expressing recombinant polypeptide. Preferably, control experiments are carried out on cells which do not express the polypeptide of the invention to establish whether the observed responses are the result of activation of the polypeptide.

The binding of a test substance to a polypeptide of the invention can be determined directly. For example, a radiolabelled test substance can be incubated with the polypeptide of the invention and binding of the test substance to the polypeptide can be monitored. Typically, the radiolabelled test substance can be incubated with cell membranes containing the polypeptide until equilibrium is reached. The membranes can then be separated from a non-bound test substance and dissolved in scintillation fluid to allow the radioactive content to be determined by scintillation counting. Non-specific binding of the test substance may also be determined by repeating the experiment in the presence of a saturating concentration of a non-radioactive ligand.

Assays may be carried out using cells expressing HIPHUM 0000037, and incubating such cells with the test substance optionally in the presence of HIPHUM 0000037 ligand. Alternatively an antibody may be used to complex HIPHUM 0000037 and thus mediate HIPHUM 0000037 activity. Test substances may then be added to assess the effect on such activity. Cells expressing HIPHUM 0000037 constitutively may be provided for use in assays for HIPHUM 0000037 function. Such constitutively expressed HIPHUM 0000037 may demonstrate HIPHUM 0000037 activity in the absence of ligand binding. Additional test substances may be introduced in any assay to look for inhibitors of ligand binding or inhibitors of HIPHUM 0000037-mediated activity.

In preferred aspects, a host cell is provided expressing the polypeptide and containing a G-protein coupled pathway responsive reporter construct. The host cell is treated with a substance under test for a defined time. The expression of the reporter gene, such as SP alkaline phosphatase or luciferase is assayed. The assay enables determination of whether the compound modulates the induction of the G-protein coupled pathway by HIPHUM 0000037 in target cells.

Assays may also be carried out to identify modulators of receptor-shedding. A polypeptide of the invention can be cleaved from the cell surface. Shedding the receptor would act to down regulate receptor signalling. Thus, cell-based assays may be used to screen for compounds which promote or inhibit receptor-shedding.

Assays may also be carried out to identify substances which modify HIPHUM 0000037 receptor expression, for example substances which up- or down-regulate expression. Such assays may be carried out for example by using antibodies for HIPHUM 0000037 to monitor levels of HIPHUM 0000037 expression. Other assays which can be used to monitor the effect of a test substance on HIPHUM 0000037 expression include using a reporter gene construct driven by the HIPHUM 0000037 regulatory sequences as the promoter sequence and monitoring for expression of the reporter polypeptide. Further possible assays could utilise membrane fractions from overexpression of HIPHUM 0000037 polypeptide either in X laevis oocytes or cell lines such as HEK293, CHO, COS7 and HeLa cells and assessment of displacement of a radiolabelled ligand.

Additional control experiments may be carried out. Assays may also be carried out using known ligands of other P2Y receptors to identify ligands which are specific for polypeptides of the invention. Preferably, the assays of the invention are carried out under conditions which would result in G-protein coupled pathway mediated activity in the absence of the test substance, to identify inhibitors or activators of P2Y-like receptor mediated activity, or agents which inhibit ligand-induced P2Y-like receptor activity. An assay of the invention may be carried out using a known P2Y receptor agonist or P2Y receptor antagonist to provide a comparison with a compound under test.

Typically, receptor activity can be monitored indirectly for example by measuring a G-protein coupled readout. G-protein coupled readout can typically be monitored using an electrophysiological method to determine the activity of G-protein regulated Ca²⁺ or K⁺ channels or by using a fluorescent dye to measure changed in intracellular Ca²⁺ levels. The receptor could be coupled to Gs, Gq, Gi and/or Go. Thus cAMP or GTP(S levels or activity, calcium mobilization, inositol triphosphate generation and protein kinase C activation may be monitored.

Following P2Y-like receptor stimulation, cyclic AMP accumulation can be measured for example in forskolin stimulated CHO cells transformed with the HIPHUM 0000037 receptor either directly, or indirectly by monitoring the expression of cotransfected reporter gene, the expression of which will be controlled by cyclic AMP response elements.

Xenopus dermal melanophores aggregate or disperse pigment in response to the activation or inhibition of G-protein coupled receptors. This feature can be exploited as an assay for receptor activation or inhibition if a specific G-protein coupled receptor is exogenously expressed.

HIPHUM 0000037 receptor is likely to couple to G-protein with consequent hydrolysis of GTP. Accumulation of a labelled GTP stable analogue can be measured utilising membrane fractions from overexpression of HIPHUM 0000037 receptor either in X laevis oocytes or cell lines such as HEK293, CHO, COS7, HeLa on exposure to agonist ligand.

G-protein coupled receptors have been shown to activate MAPK signalling pathways. Cell lines overexpressing the P2Y-like receptor of the invention with MAPK reporter genes may be utilised as assays for receptor activation or inhibition. The P2Y-like receptor of the invention may be heterologously expressed in modified yeast strains containing multiple reporter genes, such as FUS1-HIS3 and FUS1-lacZ, each linked to an endogenous MAPK cascade-based signal transduction pathway. This pathway is normally linked to pheromone receptors, but can be coupled to foreign receptors by replacement of the yeast G-protein with yeast/mammalian G protein chimeras. Strains may also contain two further gene deletions, i.e. deletions of SST2 and FAR1, to potentiate the assay. Ligand activation of the heterologous receptor can be monitored using the reporter genes, for example either as cell growth in the absence of histidine or with a substrate of beta-galactosidase (lacZ).

Suitable test substances which can be tested in the above assays include combinatorial libraries, defined chemical entities and compounds, peptide and peptide mimetics, oligonucleotides and natural product libraries, such as display (e.g. phage display libraries) and antibody products.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show inhibition or activation tested individually. Test substances may be used at a concentration of from 1 nM to 1000 μM, preferably from 1 μM to 100 μM, more preferably from 1 μM to 10 μM. Preferably, the activity of a test substance is compared to the activity shown by a known activator or inhibitor. A test substance which acts as an inhibitor may produce a 50% inhibition of activity of the receptor. Alternatively a test substance which acts as an activator may produce 50% of the maximal activity produced using a known activator.

Another aspect of the present invention is the use of polynucleotides encoding the HIPHUM 0000037 polypeptides of the invention to identify mutations in HIPHUM 0000037 genes which may be implicated in human disorders. Identification of such mutations may be used to assist in diagnosis or susceptibility to such disorders and in assessing the physiology of such disorders. Polynucleotides may also be used in hybridisation studies to monitor for up- or down-regulation of HIPHUM 0000037 expression. Polynucleotides such as SEQ ID NO: 1 or SEQ ID NO: 3 or fragments thereof may be used to identify allelic variants, genomic DNA and species variants.

The present invention provides a method for detecting variation in the expressed products encoded by HIPHUM 0000037 genes. This may comprise determining the level of an HIPHUM 0000037 expressed in cells or determining specific alterations in the expressed product. Sequences of interest for diagnostic purposes include, but are not limited to, the conserved portions as identified by sequence similarity and conservation of intron/exon structure. The diagnosis may be performed in conjunction with kindred studies to determine whether a mutation of interest co-segregates with disease phenotype in a family.

Diagnostic procedures may be performed on polynucleotides isolated from an individual or alternatively, may be performed in situ directly upon tissue sections (fixed and/or frozen) of patient tissue obtained from biopsies or resections, such that no nucleic acid purification is necessary. Appropriate procedures are described in, for example, Nuovo, G. J., 1992, “PCR In Situ Hybridization: Protocols And Applications”, Raven Press, NY). Such analysis techniques include, DNA or RNA blotting analyses, single stranded conformational polymorphism analyses, in situ hybridization assays, and polymerase chain reaction analyses. Such analyses may reveal both quantitative aspects of the expression pattern of a HIPHUM 0000037, and qualitative aspects of HIPHUM 0000037 expression and/or composition.

Alternative diagnostic methods for the detection of HIPHUM 0000037 nucleic acid molecules may involve their amplification, e.g. by PCR (the experimental embodiment set forth in U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. 15 USA 86:1173-1177), Q-Beta Replicase (Lizardi et al., 1988, Bio/Technology 6:1197) or any other nucleic acid amplification method, followed by the detection of the amplified molecules using techniques well known to those of skill in the art. These detection schemes are especially useful for the detection of nucleic acid molecules if such molecules are present in very low numbers.

Particularly suitable diagnostic methods are chip-based DNA technologies such as those described by Hacia et al., 1996, Nature Genetics 14:441-447 and Shoemaker et al., 1996, Nature Genetics 14:450-456. Briefly, these techniques involve quantitative methods for analyzing large numbers of nucleic acid sequence targets rapidly and accurately. By tagging with oligonucleotides or using fixed probe arrays, one can employ chip technology to segregate target molecules as high density arrays and screen these molecules on the basis of hybridization.

Following detection, the results seen in a given patient may be compared with a statistically significant reference group of normal patients and patients that have HIPHUM 0000037 related pathologies. In this way, it is possible to correlate the amount or kind of HIPHUM 0000037 encoded product detected with various clinical states or predisposition to clinical states.

Another aspect of the present invention is the use of the substances that have been identified by screening techniques referred to above in the treatment of disease states, which are responsive to regulation of P2Y receptor or immunomodulatory receptor activity. The treatment may be therapeutic or prophylactic. The condition of a patient suffering from such a disease state can thus be improved.

In particular, such substances may be used in the treatment of colon cancer or diseases of immunity or inflammation such as asthma, COPD, Crohn's disease, irritable bowel syndrome, gastroenteritis and colitis, inflammatory bowel syndrome, ulcerative colitis, rheumatoid arthritis, viral diseases, bacterial infections, autoimmune diseases, dermatitis, glomerulonephritis allergies, allergic rhinitis, inflammatory pain and general inflammation such as tendonitis, polymyositis, or prostatitis.

Additional disease states that may be treated include cancer, diseases related to angiogenesis such as diabetic retinopathy, psoriasis, hemangiomas, arthritis, ischemic heart disease or peripheral vascular disease, urinary incontinence—urge, stress or mixed, osteoporosis, osteoarthritis, obesity and non-insulin-dependent diabetes mellitus.

Substances identified according to the screening methods outlined above may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The substances may be administered by enteral or parenteral routes such as via oral, buccal, anal, pulmonary, intravenous, intra-arterial, intramuscular, intraperitoneal, topical or other appropriate administration routes.

A therapeutically effective amount of a modulator is administered to a patient. The dose of a modulator may be determined according to various parameters, especially according to the substance used; the age, weight and condition of the patient to be treated; the route of administration; and the required regimen. A physician will be able to determine the required route of administration and dosage for any particular patient. A typical daily dose is from about 0.1 to 50 mg per kg of body weight, according to the activity of the specific modulator, the age, weight and conditions of the subject to be treated, the type and severity of the degeneration and the frequency and route of administration. Preferably, daily dosage levels are from 5 mg to 2 g.

Nucleic acid encoding HIPHUM 0000037 or a variant thereof which inhibits HIPHUM 0000037 activity may be administered to the mammal. Nucleic acid, such as RNA or DNA, and preferably, DNA, is provided in the form of a vector, such as the polynucleotides described above, which may be expressed in the cells of the mammal.

Nucleic acid encoding the polypeptide may be administered by any available technique. For example, the nucleic acid may be introduced by needle injection, preferably intradermally, subcutaneously or intramuscularly. Alternatively, the nucleic acid may be delivered directly across the skin using a nucleic acid delivery device such as particle-mediated gene delivery. The nucleic acid may be administered topically to the skin, or to mucosal surfaces for example by intranasal, oral, intravaginal or intrarectal administration.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

The following Examples illustrate the invention.

### EXAMPLE 1

### Characterisation of the Sequence

A P2Y-like receptor, designated as HIPHUM 0000037 has been identified. The nucleotide and amino acid sequences of the receptor have been determined. These are set out below in SEQ ID NOs: 1 and 2. The nucleotide and amino acid sequences of a variant of the receptor comprising an extended 5′ region have also been determined. These are set out below in SEQ ID Nos: 3 and 4.

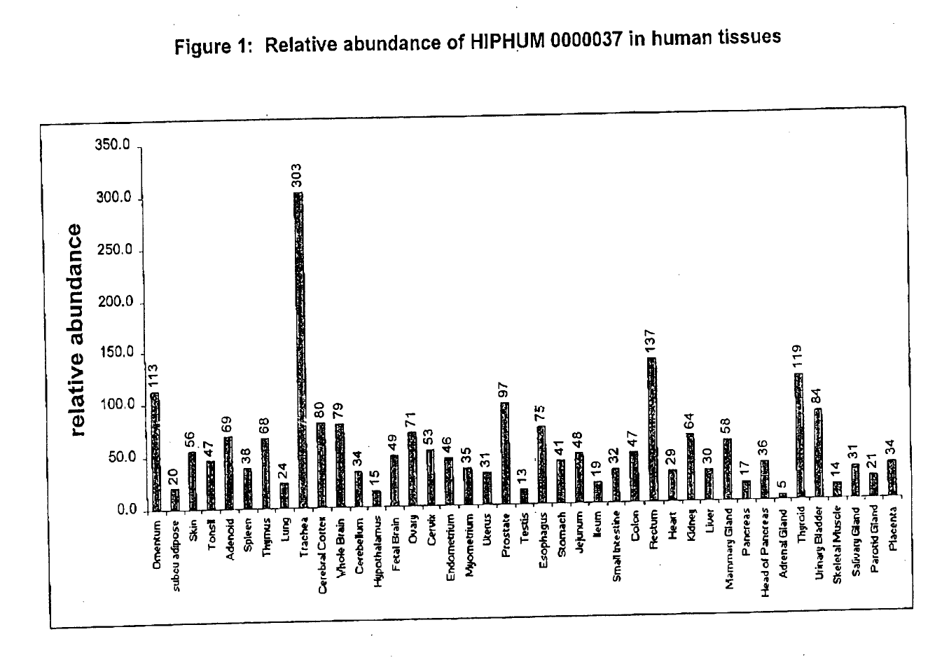

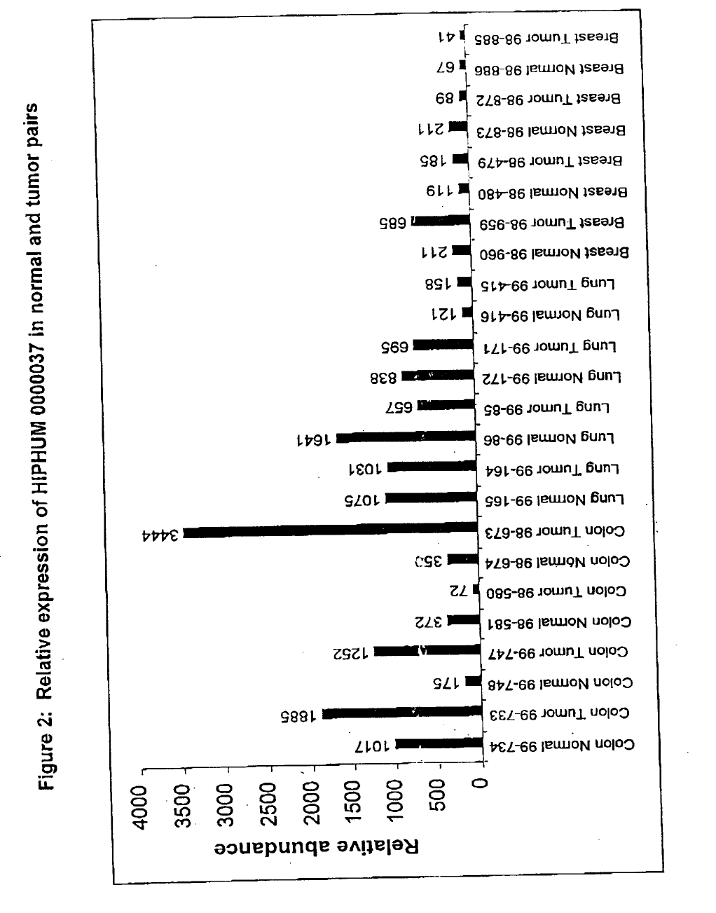

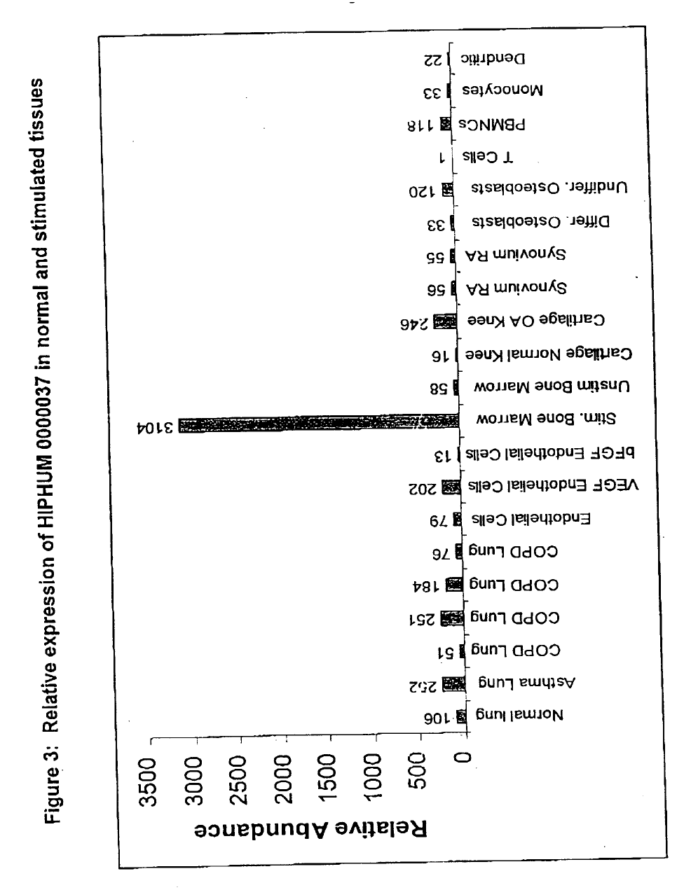

Suitable primers and probes were designed and used to analyse tissue expression. Expression of HIPHUM 0000037 was found to be ubiquitous (FIG. 1). This pattern may be explained by the expression of the receptor in some immune cells (monocytes and dendritic cells), adipose tissue and in endothelial cells (FIG. 3). Growth factor stimulation of bone marrow cells upregulated the expression of this receptor. Expression was 15 fold higher in osteoarthritic than normal cartilage and was down-regulated in differentiated osteoblasts (FIG. 3). FIG. 2 shows expression of the receptor in normal and tumor pairs. Receptor expression was up-regulated in 3 colon tumors vs. paired normal tissues. Thyroid and urinary bladder expression was also higher than other tissues.

### EXAMPLE 2

### Screening for Substances which Exhibit Protein Modulating Activity

Mammalian cells, such as HEK293, CHO and COS7 cells, over-expressing a polypeptide of the invention are generated for screening purposes. 96 and 384 well plate, high throughput screens (HTS) are employed using fluorescence based calcium indicator molecules, including but not limited to dyes such as Fura-2, Fura-Red, Fluo 3 and Fluo 4 (Molecular Probes). Secondary screening involves the same technology. Tertiary screens involve the study of modulators in rat, mouse and guinea-pig models of disease relevant to the target.

A brief screening assay protocol is as follows:

Mammalian cells stably over-expressing a polypeptide of the invention are cultured in black wall, clear bottom, tissue culture-coated, 96 or 384 well plates with a volume of 100 82 l cell culture medium in each well 3 days before use in a FLIPR (Fluorescence Imaging Plate Reader—Molecular Devices). Cells are incubated with 4 μM FLUO-3AM at 30° C. in 5%CO₂ for 90 mins and then washed once in Tyrodes buffer containing 3 mM probenecid. Basal fluorescence is determined prior to addition of test substances. The polypeptide is activated upon the addition of a known agonist. Activation results in an increase in intracellular calcium which can be measured directly in the FLIPR. For antagonist studies, substances are preincubated with the cells for 4 minutes following dye loading and washing and fluorescence measured for 4 minutes. Agonists are then added and cell fluorescence measured for a further 1 minute.

Assays may also be carried out as follows:

Gs-coupled receptors are expressed and assayed in mammalian cells which express the 6× CRE-luciferase reporter gene such as CHO cells. Gq-coupled and Gi-coupled receptors are expressed and assayed in mammalian cells which express the Gal4/Elk-1 chimeric protein and 5× UAS-luciferase reporter gene. Cells are propagated in either in suspension or adherent cultures.

For adherent culture, cells are propagated in T225 flasks in DMEM/F12 containing 5% fetal bovine serum and 1 mM glutamine. Forty-eight hours prior to assay, cells are harvested with 2 ml of 0.05% trypsin, washed with complete medium and plated at a concentration of 4,000 cells/well in complete medium. Sixteen hours prior to the assay, the medium is removed from the cells and replaced with 90 μl/well of serum-free DMEM/F12. At the time of the assay, test substances are added to the wells at a final concentration of 10 μM and the plates are incubated for four hours at 37° C. in a cell culture incubator. The medium is aspirated by vacuum followed by the addition of 50 μl of a 1:1 mixture of LucLite™ and dPBS/1 mM CaCl₂/1 mM MgCl₂. Plates are sealed and subjected to dark adaptation at room temperature for 10 minutes before luciferase activity is quantitated on a TopCount™ microplate scintillation counter (Packard) using 3 seconds/well count time.

For suspension cultures, cells are propagated in Excel 301 medium containing 5% FBS and 2 mM glutamine at a minimum of 1×10⁵ cells/ml for one week. Sixteen hours prior to an assay, cells are removed from suspension by centrifugation and resuspended in serum-free Excel 301 at a concentration of 1×10⁶ cells/ml. At the time of assay, the cells are resuspended in serum-free DMEM/F12 at a concentration of 50,000 cells/ml. 100 μl/well or 50 μl/well of this suspension is pipetted into black 96-well or 384-well plates, respectively. The 96-well and 384-well plate contained 1 ul or 0.5 μl of agonist compounds in 100% DMSO at a final concentration of 10 μM. A Multidrop S20 cell dispenser is used to dispense cells into either 96-or 384-well plates. The reminder of the assay is the same as described for adherent culture above.

Xenopus Oocyte Expression

Adult female Xenopus laevis (Blades Biologicals) are anaesthetised using 0.2% tricaine (3-aminobenzoic acid ethyl ester), killed and the ovaries rapidly removed. Oocytes are then de-folliculated by collagenase digestion (Sigma type I, 1.5 mg ml⁻¹) in divalent cation-free OR2 solution (82.5 mM NaCl, 2.5 mM KCl, 1.2 mM NaH₂PO₄, 5 mM HEPES; pH 7.5 at 25° C.). Single stage V and VI oocytes are transferred to ND96 solution (96 mM NaCl, 2 mM KCl, 1 mM MgCl₂, 5 mM HEPES, 2.5 mM sodium pyruvate; pH 7.5 at 25° C.) which contains 50 μg ml⁻¹ gentamycin and stored at 18° C.

The P2Y-like receptor (in pcDNA₃, invitrogen) is linearised and transcribed to RNA using T7 (Promega Wizard kit). m′G(5′)pp(5′)GTP capped cRNA is injected into oocytes (20-50 ng per oocyte) and whole-cell currents are recorded using two-microelectrode voltage-clamp (Geneclamp amplifier, Axon instruments Inc.) 3 to 7 days post-RNA injection. Microelectrodes have a resistance of 0.5 to 2 MΩ when filled with 3 M KCl.

Melanophore screens may be carried out as follows:

Modified or unmodified receptors are expressed in melanophores using appropriate vector constructs including pJG3.6. The expressed receptors are then screened for Gs, Gq, Gi or Go activity. When a ligand binds to a Gs-coupled receptor, it activates adenylyl cyclase that in turn activates protein kinase A. This results in the initiation of phosphorylation events that cause the melanosomes to disperse. When a Gi-coupled receptor is activated, it inhibits adenylyl cyclase which in turn reverses the pigment dispersion process to result in aggregation. When a G_{q}-coupled receptor is activated, it activates phospholipase C, which in turn activates protein kinase C. This results in the initiation of phosphorylation events to cause melanosome dispersion. The expressed receptors can be screened in agonist, antagonist or constitutive modes using bead-based lawn format or 96-well, 384-well or 1536-well formats.

Melanophores are grown in conditioned fibroblast medium (CFM) at room temperature. After harvesting the cells with trypsin/EDTA, approximately 6 to 10 million cells are electroporated with relevant receptor-expression vectors at 475 V, 425 μFd, 720 ohms. The transfected cells are then plated into T225 flasks and are incubated for 24 hours. Cells are then harvested and plated into assay plates and incubated for 24 hours. Test substances are added to wells at 10 uM final concentration and 30-120 minutes later the dispersion or aggregation is measured using an SLT Spectra plate reader. For dispersion assays, cells are first treated with 2 nM melatonin in assay buffer (0.7× L15/0.1% BSA) for 60 minutes before addition of test compounds. For aggregation assays, CFM is replaced with the assay buffer and cells are incubated for 60 minutes before addition of test compounds.

1. An isolated P2Y-like receptor polypeptide comprising
(i) the amino acid sequence of SEQ ID NO: 2 or SEQ ID NO: 4 or
(ii) a variant thereof which shows immunomodulatory activity or
(iii) a fragment of (i) or (ii) which shows immunomodulatory activity.

2. A polypeptide according to claim 1 wherein the variant (ii) has at least 80% identity to the amino acid sequence of SEQ ID NO: 2 or SEQ ID NO: 4.

3. A polynucleotide encoding a polypeptide according to claim 1 or 2.

4. A polynucleotide according to claim 3 which is a cDNA sequence.

5. A polynucleotide encoding a P2Y-like receptor polypeptide which shows immunomodulatory activity which polynucleotide comprises:
(a) the nucleic acid sequence of SEQ ID NO: 1 or SEQ ID NO: 3 and/or a sequence complementary thereto;
(b) a sequence which hybridises under stringent conditions to a sequence as defined in (a);
(c) a sequence that is degenerate as a result of the genetic code to a sequence as defined in (a) or (b); or
(d) a sequence having at least 80% identity to a sequence as defined in (a), (b) or (c).

6. An expression vector comprising a polynucleotide according to any one of claims 3 to 5.

7. A host cell comprising an expression vector according to claim 6.

8. An antibody specific for a polypeptide according to claim 1 or 2.

9. A method for the identification of a substance that modulates P2Y-like receptor activity and/or expression, which method comprises:
(i) contacting a test substance and a polypeptide according to claim 1 or 2, a polynucleotide according to any one of claims 3 to 5, an expression vector according to claim 6 or a host cell according to claim 7, and
(ii) determining the effect of the test substance on the activity and/or expression of the said polypeptide or the polypeptide encoded by said polynucleotide, thereby to determine whether the test substance modulates P2Y-like receptor activity and/or expression.

10. A method according to claim 9 wherein the polypeptide is expressed in a cell.

11. A substance which modulates P2Y receptor activity and which is identifiable by a method according to claim 9 or 10.

12. A method of treating a subject having a disorder that is responsive to P2Y-like receptor modulation, which method comprises administering to said subject an effective amount of a substance according to claim 11.

13. A method according to claim 12 wherein the disorder is a disease of immunity or inflammation.

14. Use of a substance as defined in claim 11 in the manufacture of a medicament for treatment or prophylaxis of a disorder that is responsive to stimulation or modulation of P2Y-like receptor activity.

15. A use according to claim 14 wherein the disorder is a disease of immunity or inflammation.

16. A method of producing a polypeptide according to claim 1 or 2, which method comprises maintaining a host cell as defined in claim 7 under conditions suitable for obtaining expression of the polypeptide and isolating the said polypeptide.

17. An isolated polynucleotide comprising a polynucleotide whose sequence is set forth in SEQ ID NO: 1 or SEQ ID NO:3.

18. The isolated polynucleotide of claim 1 wherein the polynucleotide consists of a polynucleotide whose sequence is set forth in SEQ ID NO: 1 or SEQ ID NO:3.

19. The isolated polynucleotide of claim 1 wherein the polynucleotide encodes a polypeptide whose sequence is set forth in SEQ ID NO:2 or SEQ ID NO:4.

20. An isolated polynucleotide that encodes a polypeptide comprising an amino acid sequence set forth in SEQ ID NO:2 or SEQ ID NO:4.

21. An isolated polypeptide comprising a polypeptide whose sequence is set forth in SEQ ID NO:2 or SEQ ID NO:4.

22. An isolated polypeptide of claim 5 consisting of a polypeptide whose sequence is set forth in SEQ ID NO:2.

23. A vector comprising the isolated polynucleotide of claim 4.

24. An isolated host cell comprising the vector of claim 8.

25. A process for producing a P2Y-like receptor polypeptide comprising culturing a host cell of claim 8 under conditions sufficient for the production of said polypeptide
